(19) United States
(12) Patent Application Publication
(10) Pub. No.: US 2024/0425472 A1
(43) Pub. Date: Dec. 26, 2024

(54) METHOD FOR PRODUCING TRIOXANE OR METHYLAL

(71) Applicant: ASAHI KASEI KABUSHIKI KAISHA, Tokyo (JP)

(72) Inventors: Kunitaka KURODA, Tokyo (JP); Makoto DOKI, Tokyo (JP)

(73) Assignee: ASAHI KASEI KABUSHIKI KAISHA, Tokyo (JP)

(21) Appl. No.: 18/749,269

(22) Filed: Jun. 20, 2024

(30) Foreign Application Priority Data
Jun. 22, 2023 (JP) 2023-102790
Nov. 2, 2023 (JP) 2023-188853

(51) Int. Cl. C07D 323/06; C07C 41/56

(52) U.S. Cl. CPC C07D 323/06; C07C 41/56

(57) ABSTRACT

A method for producing trioxane or methylal is provided in which the decrease in the intended reaction activity is suppressed and the volume change of the styrene-based resin is suppressed even over prolonged use. A method for producing trioxane or methylal includes the step of producing trioxane or methylal by reacting formaldehyde in the presence of a styrene-based resin having electron withdrawing groups. A solid-state ¹³C-NMR spectrum of the styrene-based resin has Peak 1 having a peak top in the range of 32 to 57 ppm and Peak 2 having a peak top in the range of 120 to 137 ppm. The area ratio (A1/A2) of the area A1 of Peak 1 to the area A2 of Peak 2 is 1.00 to 1.40.

### TECHNICAL FIELD

The present disclosure relates to a method for producing trioxane or methylal.

### BACKGROUND

Trioxane is a cyclic trimer of formaldehyde and is mainly used as a raw material for the production of polyoxymethylene. Methylal is produced by reacting formaldehyde with methanol, and oxidizing methylal gives formaldehyde in a high concentration.

The methods for producing trioxane and methylal both use formaldehyde as a raw material and employ an acid catalyst (e.g., sulfuric acid, benzenesulfonic acid, toluenesulfonic acid, silica-alumina, zeolite, cation exchange resin, etc.), thus sharing common issues.

Although common industrial methods for producing trioxane commonly utilize sulfuric acid, sulfuric acid poses problems such as corrosion of apparatuses, etc., generation of large amounts of by-products, and the production of paraformaldehyde.

As a solution to these problems, methods through utilizing styrene-based resins, particularly cation exchange resins, have been proposed. PTL 1 discloses a method for producing trioxane from formaldehyde using a cation exchange resin having a —SO₃H group as an exchange group.

Furthermore, PTL 2 discloses a method by utilizing a macroporous cation exchange resin as a method with a high selectivity for trioxane.

### CITATION LIST

### Patent Literature

PTL 1: JP S40-12794 B
PTL 2: JP S64-11025 B

### SUMMARY

When trioxane is produced using a cation exchange resin, it is preferable to bring an aqueous solution of formaldehyde into contact with the cation exchange resin for efficient production of trioxane.

Bringing the aqueous solution of formaldehyde into contact with the cation exchange resin to continue the reaction, however, can lead to the release of sulfonic acid groups from the resin, causing a decrease in ion exchange capacity and consequently lowering the synthetic activity for trioxane.

Furthermore, continuing the reaction for a long time can cause the cation exchange resin to swell, resulting in an increase in volume. Consequently, overpacking of the resin can occur depending on the charge volume of the cation exchange resin.

Therefore, the present disclosure is directed to providing a method for producing trioxane or methylal wherein the decrease in intended reaction activity is suppressed and the volume change of the styrene-based resin is suppressed even over prolonged use.

As a result of our diligent study, the present inventors have discovered that the aforementioned problems can be solved by setting the degree of cross-linking of a styrene-based resin within a specified range, leading to the completion of the present disclosure.

Specifically, the present disclosure provides the following:

(1)

A method for producing trioxane or methylal comprising the step of
reacting formaldehyde in the presence of a styrene-based resin having an electron-withdrawing group to produce trioxane or methylal,
wherein a solid-state ¹³C-NMR spectrum of the styrene-based resin has Peak 1 having a peak top in a range of 32 to 57 ppm and Peak 2 having a peak top in a range of 120 to 137 ppm, and
an area ratio (A1/A2) of an area A1 of Peak 1 to an area A2 of Peak 2 is 1.00 to 1.40.
   (2)

The method for producing trioxane or methylal according to (1), wherein the styrene-based resin is a cation exchange resin.

(3)

The method for producing trioxane or methylal according to (1) or (2), wherein the styrene-based resin comprises a constituent unit represented by the following formula (I) and a constituent unit represented by the following formula (II):

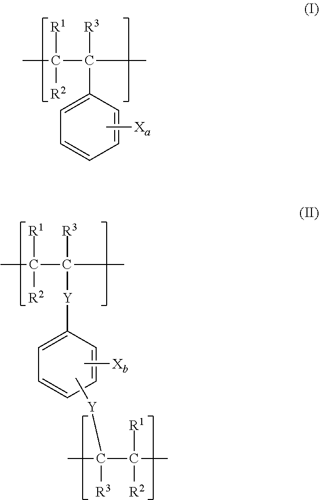

in the formulae (I) and (II),
R¹, R², and R³ are each independently a hydrogen atom, an alkyl group, an aryl group, or a halogen atom;
X is an electron-withdrawing group, and the plurality of X may be different from each other;
Y is an alkylene group, an arylene group, or a single bond;
a is each independently an integer from 1 to 5;
b is each independently an integer from 1 to 4.
   (4)

The method for producing trioxane or methylal according to any one of (1) to (3), wherein
a catalyst charge tank charged with the styrene-based resin is used, and
a charge ratio of the styrene-based resin in the catalyst charge tank is 20 to 90% of a volume of a chargeable portion in the catalyst charge tank.
   (5)

The method for producing trioxane or methylal according to any one of (1) to (4), wherein the reaction of formaldehyde in the step is a continuous reaction.

(6)

The method for producing trioxane or methylal according to (4), wherein a residence time of formaldehyde in the catalyst charge tank is 0.01 to 50 minutes.

(7)

The method for producing trioxane or methylal according to any one of (4) to (6), further comprising a step (P) of passing an aqueous solution of formaldehyde in a volume equal to or greater than a charge volume of the styrene-based resin, through the catalyst charge tank before the step.

According to the present disclosure, it is possible to produce trioxane or methylal while suppressing the decrease in intended reaction activity and suppressing the volume change of the styrene-based resin even over prolonged use.

### BRIEF DESCRIPTION OF THE DRAWINGS

In the accompanying drawings:

FIG. 1 is the solid-state ¹³C-NMR spectrum of the styrene-based resin of Production Example 5; and

FIG. 2 is a flow diagram schematically illustrating one example of the trioxane production process.

### DETAILED DESCRIPTION

Hereinafter, an embodiment for embodying the present disclosure (hereinafter referred to merely as “the present embodiment”) will be described in detail. Note that the present disclosure is not limited by the description given below, and may be implemented with various changes or modifications that are within the essential scope thereof.

In the present embodiment, a method includes the step of reacting formaldehyde in the presence of a styrene-based resin having an electron-withdrawing group to produce trioxane or methylal,
wherein a solid-state ¹³C-NMR spectrum of the styrene-based resin has Peak 1 having a peak top in the range of 32 to 57 ppm and Peak 2 having a peak top in the range of 120 to 137 ppm, and
the area ratio (A1/A2) of the area A1 of Peak 1 to the area A2 of Peak 2 is 1.00 to 1.40. An area ratio (A1/A2) of 1.00 to 1.40 has superiority in terms of reaction activity and suppression of the volume change ratio.

In one preferred embodiment, the styrene-based resin having the electron-withdrawing group contains the constituent units represented by the following formulae (I) and (II).

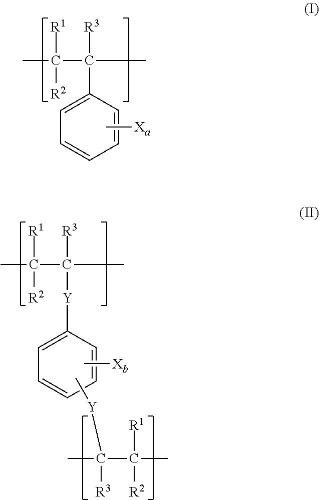

In the formula (I),
R¹, R², and R³ are each independently a hydrogen atom, an alkyl group, an aryl group, or a halogen atom;
X is an electron-withdrawing group, and the plurality of X may be different from each other;
a is each independently an integer from 1 to 5.

In the formula (II),
R¹, R², and R³ are each independently a hydrogen atom, an alkyl group, an aryl group, or a halogen atom;
X is an electron-withdrawing group, and the plurality of X may be different from each other;
Y is an alkylene group, an arylene group, or a single bond;
a is each independently an integer from 1 to 5; and
b is each independently an integer from 1 to 4.

Examples of the alkyl group in R¹, R², and R³ in the formulae (I) and (II) include linear, branched-chain, and cyclic alkyl groups. Examples of linear alkyl groups include, for example, methyl, ethyl, propyl, and butyl groups. Examples of branched-chain alkyl groups include, for example, isopropyl, isobutyl, and tert-butyl groups. Examples of cyclic alkyl groups include, for example, cyclopropyl, cyclobutyl, cyclopentyl, and cyclohexyl groups.

Examples of the aryl group in R¹, R², and R³ in the formulae (I) and (II) include, for example, phenyl, 1-naphthyl, 2-naphthyl, anthryl, and phenanthryl groups.

Examples of the halogen atom in R¹, R², and R³ in the formulae (I) and (II) include, for example, fluorine, chlorine, bromine, and iodine atoms.

The alkyl and aryl groups in R¹, R², and R³ in the formulae (I) and (II) may or may not have a substituent. Examples of the substituent include, for example, alkyl groups and halogen atoms as exemplified for R¹.

In a preferred embodiment, R¹, R², and R³ are hydrogen atoms.

Examples of the electron-withdrawing groups X in the formulae (I) and (II) include, for example, a sulfonic acid group, carboxylic acid group, nitro group, cyano group, phosphoric acid group, fluorine atom, chlorine atom, bromine atom, and iodine atom.

Examples of alkylene groups include linear alkylene groups such as methylene and ethylene groups, branched-chain alkylene groups, and cyclic alkylene groups. Alkylene groups may or may not have a substituent. Examples of the substituent include, for example, alkyl groups and halogen atoms as exemplified for R¹.

The arylene group in Y in the formula (II) is, for example, a phenylene group. The arylene group may or may not have a substituent. Examples of the substituent include, for example, alkyl groups and halogen atoms as exemplified for R¹.

When Y in the formula (II) is a single bond, a carbon in the main chain is directly bonded to a carbon in the aromatic ring.

In a preferred embodiment, Y is a single bond.

Without limitation, the ends of the compound containing the constituent units represented by the formulae (I) and (II) may have the substituents mentioned above or may have substituents derived from a polymerization initiator.

A solid-state ¹³C-NMR spectrum of the styrene-based resin used in the present embodiment has Peak 1 having a peak top in the range of 32 to 57 ppm and Peak 2 having a peak top in the range of 120 to 137 ppm, and the area ratio of the area A1 of Peak 1 to the area S2 of Peak 2 (A1/A2) is 1.00 to 1.40. In one preferred embodiment, the area ratio (A1/A2) is 1.39 or less, 1.35 or less, or 1.30 or less. In another preferred embodiment, the area ratio (A1/A2) is 1.02 or more, 1.05 or more, 1.10 or more, or 1.12 or more. In one embodiment, the area ratio (A1/A2) is 1.02 to 1.39.

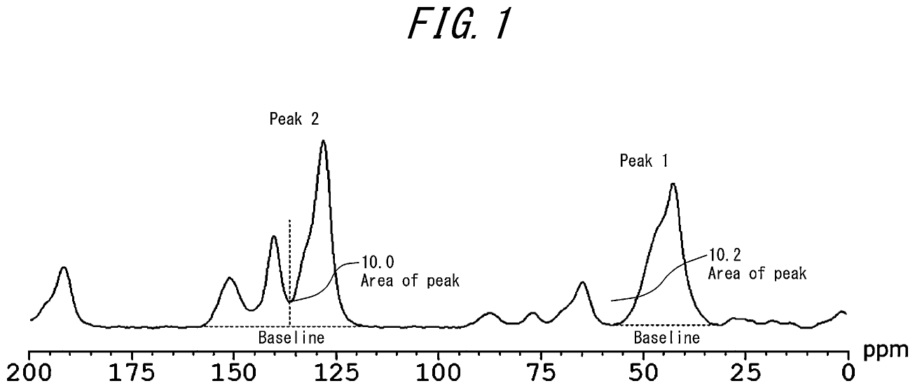

FIG. 1 is the solid-state ¹³C-NMR spectrum of the styrene-based resin of Production Example 5. The horizontal axis represents the chemical shift values (ppm) and the vertical axis represents the relative intensities of the signals. Chemical shift values were obtained using glycine as an external standard.

As depicted in FIG. 1, the peak having a peak top in the range of 32 to 57 ppm is referred to as “Peak 1”, and the peak having a peak top in the range of 120 to 137 ppm is referred to as “Peak 2”. Here, it is considered that Peak 1 is derived from aliphatic carbons, and Peak 2 is derived from aromatic carbons (which may include substituted vinyl group carbons on aromatic rings).

A1 is the area of the spectral region bounded by Peak 1 and the line (baseline) connecting the flat spectral regions free of peaks on either side of Peak 1. Similarly, A2 is the area of the spectral region bounded by Peak 2 and the line (baseline) connecting the flat spectral regions free of peaks on either side of Peak 2. The area ratio (A1/A2) can be determined as the ratio of the peak areas of A1 to A2. For example, when the area A1 of Peak 1 is 10.2 and the area A2 of Peak 2 is 10.0, the area ratio (A1/A2) is 10.2/10.0=1.02.

One method to control the area ratio (A1/A2) is to adjust the degree of cross-linking of the styrene-based resin. For example, methods such as adjusting the amount of the cross-linking agent added during polymerization, adjusting the polymerization temperature during the polymerization step, particularly adjusting the stirring speed or forcing polymerization to promote when the polymerization rate slows down at the later stage of the polymerization can be mentioned. Additionally, another method involves subjecting the styrene-based resin to a certain treatment before actually using it as a catalyst.

When adjusting the polymerization temperature of the styrene-based resin, it is preferable to set the polymerization temperature from 78 [symbol] C to 100 [symbol] C, and more preferable from 86 [symbol] C to 92 [symbol] C, for example.

Means to promote polymerization at the later stage of the polymerization include raising the polymerization temperature or adding an additional initiator at the later stage of the polymerization. The polymerization temperature at the later stage of the polymerization is preferably from 88 [symbol] C to 100 [symbol] C, and more preferably from 92 [symbol] C to 95 [symbol] C.

The stirring speed is set to, for example, preferably from 20 rpm to 200 rpm, and more preferably from 50 rpm to 150 rpm.

One example of a method involving subjecting the styrene-based resin to a certain treatment before actually using it as a catalyst is immersing the styrene-based resin in a formaldehyde liquid. From the time of production of the styrene-based resin until charging it into the catalyst charge tank as a catalyst, electron-withdrawing groups have detached from the styrene-based resin. The generation of by-products is likely to be suppressed by removing such electron-withdrawing groups to the outside of the reaction system for obtaining trioxane or methylal. Hence, in view of the reactivity change ratio, it is preferable that an aqueous solution of formaldehyde or a mixture of water, formaldehyde, and methanol is passed through the catalyst charge tank after the styrene-based resin is charged into the catalyst charge tank and before the step of reacting formaldehyde, for example.

In the case of producing trioxane, the formaldehyde liquid that is passed through the catalyst charge tank is an aqueous solution of formaldehyde. The concentration of formaldehyde in the aqueous solution of formaldehyde is, for example, 30 to 80 weight %, preferably 60 to 75 weight %.

In the case of producing methylal, the formaldehyde liquid that is to be passed through the catalyst charge tank is a mixture of formaldehyde and methanol. The mixing ratio formaldehyde:methanol in a molar ratio is, for example, 1:1 to 1:3, preferably 1:2.

In one embodiment, before the step of reacting formaldehyde in the presence of the styrene-based resin having electron withdrawing groups to produce trioxane or methylal, the method further includes the step (P) of passing an aqueous solution of formaldehyde in a volume equal to or greater than, but not more than ten times the charge volume of the styrene-based resin through a catalyst charge tank charged with the styrene-based resin.

In the step of reacting formaldehyde in the presence of the styrene-based resin having electron withdrawing groups to produce trioxane or methylal, it is preferable to use the catalyst charge tank charged with the styrene-based resin. The charge ratio of the styrene-based resin in the catalyst charge tank may be adjusted accordingly, and is, for example, 20 to 95% of the volume of the chargeable portion in the catalyst charge tank. In one preferred embodiment, the charge ratio is 95% or less, 92% or less, 90% or less, 85% or less, or 80% or less, in view of the reactivity change ratio. In another preferred embodiment, the charge ratio is 20% or more, 25% or more, 30% or more, 40% or more, or 50% or more. In one embodiment, the charge ratio is 50 to 92%.

The mode of reaction of the production method of the present embodiment is not particularly limited and can be, for example, the batch, semi-batch, or continuous mode. The continuous mode is preferred as the reaction mode.

The residence time of formaldehyde in the catalyst charge tank is not limited and may be from 0.01 minutes to 60 minutes, for example. In one preferred embodiment, the residence time is 50 minutes or shorter, 40 minutes or shorter, 30 minutes or shorter, 20 minutes or shorter, or 10 minutes or shorter. In another preferred embodiment, the residence time is 0.01 minutes or longer or 1 minute or longer.

In the present embodiment, an aqueous solution of formaldehyde may be used as a raw material. The concentration of formaldehyde in the aqueous solution of formaldehyde is not particularly limited and is 30 to 80 weight %, preferably 60 to 75 weight %, for example.

One example of an industrial implementation of the present embodiment is as follows.

### Production Method of Trioxane

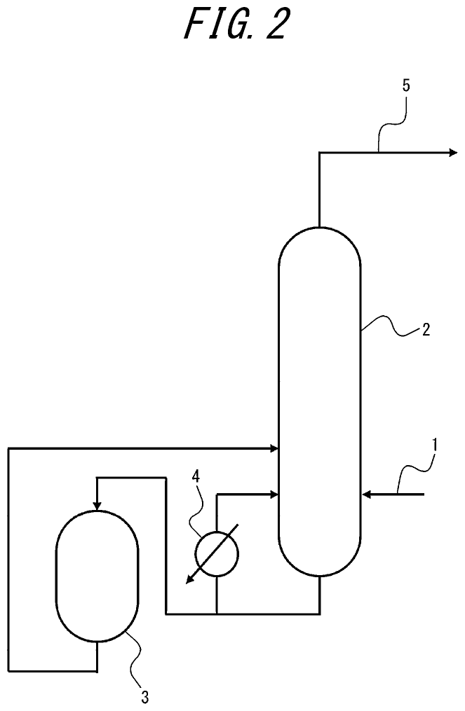

FIG. 2 is a flow diagram schematically illustrating one example of the trioxane production process. In the example depicted in FIG. 2, the raw material (aqueous solution of formaldehyde) is fed from a raw material supply line 1 to the tower bottom (bottom) of a distillation tower 2. The liquid in the distillation tower 2 is then circulated through a catalyst charge tank 3 and a reboiler 4 at a certain temperature to synthesize trioxane. Thereafter, trioxane, water, formaldehyde, and by-products are distilled through a distillation line 5 at the top of the tower. The distillate containing trioxane is then brought into contact with an organic solvent to extract trioxane into the organic solvent phase, and the extract is further purified to yield high-purity trioxane.

### Production Method of Methylal

In the production of methylal, an aqueous solution of formaldehyde and methanol is supplied as raw materials as in the production of trioxane. Methylal is synthesized by the same process as in FIG. 2 and the methylal produced is distilled from the top of the distillation tower 2.

An embodiment to embody the present disclosure has been described, but the present disclosure is not limited to the above-mentioned present embodiment. Various changes and modifications may be made without departing from the spirit of the present disclosure.

### EXAMPLES

The following provides a more detailed description of the present disclosure through specific examples and comparative examples. However, the present disclosure is not limited to the following examples. The terminology and measurement methods of the properties in the examples and comparative examples are as follows.

### (1) Peak Area Ratio (A1/A2)

The area ratio (A1/A2) was measured for a styrene-based resin used in the present embodiment by solid-state ¹³C-NMR under the following conditions.

### Pretreatment

A sample of a styrene-based resin to be measured was ground into a powder and vacuum dried at 25 [symbol] C and 0.01 MPa until the weight did not change. The sample was then packed in a solid-state NMR sample tube made of zirconia, and the measurement was performed.

Measurement conditions for solid-state ¹³C-NMR
Apparatus: Avance 500 manufactured by Bruker Japan
Observed nucleus: ¹³C
Observation frequency: 125.8 MHz
Measurement method: CP/MAS technique (pulse program cp)
¹H pulse width: 90° pulse
Contact time: 2000 μs
Waiting time: 5 s
Accumulation count: 1024 times
Measurement temperature: room temperature (25° C.)
Probe: 4-mm probe
MAS rotation speed: 8 kHz
Standard for chemical shifts: Glycine (external standard, 176.03 ppm)

The measurement conditions for gas chromatography in the evaluation of the reactivity change ratio to be described below are as follows.

### (Measurement Conditions for Gas Chromatography)

Apparatus: GC-2014S manufactured by Shimadzu Corporation
Detector: thermal conductivity detector (TCD)
Filler: Polapak T (50/80 mesh) manufactured by Nippon Waters
Inlet temperature: 230° C.
Column temperature: 125 to 180° C.
Detection temperature: 230 [symbol] C
(After holding for 8 minutes, the temperature is increased to 180 [symbol] C at 20 [symbol] C/min and held at 180 [symbol] C for 15 minutes.)
Carrier gas: hydrogen

### (2) Ratios of Changes in Characteristics Before and After Long-Term Test

### (2-1) Long-Term Test (Batch Test)

The styrene-based resin produced in each production example described later was placed into a measuring cylinder. Pure water was added until the styrene-based resin was completely immersed, and the top of the styrene-based resin was leveled horizontally at the 125 mL mark. After water was sufficiently removed from the styrene-based resin, the styrene-based resin was charged into a pressure-resistant vessel having a lining made of a polytetrafluoroethylene (PTFE) (PTFE-lined sealable heating reactor, model number: YHR-250N, capacity: 250 mL).

Subsequently, an aqueous solution of formaldehyde was then passed through a pressure-resistant container serving as a catalyst charge tank. More specifically, 50 mL of a 67-weight % aqueous solution of formaldehyde heated to 100 [symbol] C was placed in the pressure-resistant container, the content was stirred with a spatula, and the supernatant liquid was removed. This operation was repeated four times. The aqueous solution of formaldehyde was then added up to the level of the top of the styrene-based resin, and the container was sealed. The container was placed in an oven (model number: LCV-233, manufactured by ESPEC Corporation) that had been preheated to 110 [symbol] C. It was defined that the time when the container was placed in the oven was used as the starting point and that 24 hours later from the starting point would count as one day passed. After 150 days passed, the container was removed from the oven and cooled to room temperature. Subsequently, the styrene-based resin was removed from the container and rinsed with pure water. In the case of synthesizing methylal, the process was carried out in the same manner except that a mixture of formaldehyde and methanol (formaldehyde:methanol (in the molar ratio)=1:2) was used instead of the aqueous solution of formaldehyde and the preheating temperature of the oven was changed to 70 [symbol] C.

### (2-2) Volume Change Ratio (%) Before and After Long-Term Test

Before and after the long-term test, the styrene-based resin was placed in a measuring cylinder. Pure water was added until the styrene-based resin was completely immersed, and the top of the styrene-based resin was leveled horizontally. The volume of the styrene-based resin was then read from the scale on the measuring cylinder. The volume change ratio was calculated using the following formula. If the volume change ratio was 150% or less, it was determined that the production can be performed without overfilling of the styrene-based resin.

$$Volume change ratio ( \% ) = ( volume in mL of styrene - based resin after test ) / ( volume in mL of styrene - based resin before test ) \times 100$$

### (2-3) Reactivity Change Ratio Before and After Long-Term Test (%)

Before and after the long-term test, 20 mL of the styrene-based resin was measured out and charged into a glass tube. A 67-weight % aqueous solution of formaldehyde heated to 100 [symbol] C was continuously fed at 200 g/hr into a glass tube heated to 100 [symbol] C. In the case of synthesizing methylal, a mixture of formaldehyde and methanol (formaldehyde:methanol (in the molar ratio)=1:2) was continuously fed at 200 g/hr into a glass tube heated to 70 [symbol] C. Of the reaction solution after passing through the solution, 1 g was sampled, and 10 g of heated pure water was added to make a homogeneous solution. The concentration (in weight %) of the target product, i.e., trioxane or methylal, was determined by gas chromatography using the solutions before and after the long-term test. The reactivity change ratio (%) before and after the long-term test was calculated from the following formula. If the reactivity change ratio was 70% or more, it was determined that the production can be performed while the decrease in intended reaction activity is suppressed even over prolonged use.

$$Reactivity change ratio ( \% ) = concentration ( in weight \% ) of target product in the presence of a styrene - based resin after test / concentration ( in weight \% ) of target product in the presence of a styrene - based resin before test \times 100$$

### (3) Ratios of Changes in Characteristics Before and After Long-Term Operation

### (3-1) Long-Term Operation (Continuous Operation)

Of the styrene-based resin produced in each production example described later, 150 mL was measured out using a measuring cylinder and charged into a catalyst charge tank with a volume of 300 mL. Subsequently, an aqueous solution of formaldehyde in a volume equal to or greater than the charge volume was passed through the catalyst charge tank. A 67-weight % aqueous solution of formaldehyde heated to 100 [symbol] C was then fed to the bottom of an Aldershaw distillation tower (15 stages). Subsequently, the catalyst charge tank was installed on the circulation line as illustrated in FIG. 2. The bottom liquid containing the aqueous solution of formaldehyde and the synthesized trioxane was then circulated at a circulated liquid rate of 1500 g/hr. The bottom was heated with a heater to perform distillation without reflux. The distillate was then discharged at 150 g/hr and trioxane was synthesized. The amount of aqueous solution of formaldehyde supplied to the bottom was controlled so that the level of the bottom solution remained constant.

### (3-2) Volume Change Ratio (%) Before and After Long-Term (Continuous) Operation

After operating the above-described production facility for 150 days, the height from the bottom of the catalyst charge tank charged with the styrene-based resin to the top of the styrene-based resin was measured. This height was defined as the height of the charge tank for the styrene-based resin after long-term operation. The volume change ratio (%) was then calculated from the following formula.

$$Volume change ratio ( \% ) = ( height in cm of charge tank of styrene - based resin after long - term operation ) / ( height in cm of charge tank of styrene - based resin before long - term operation ) \times 100$$

### (3-3) Reactivity Change Ratio (%) Before and After Long-Term (Continuous) Operation

After operating the above-described production facility for 150 days, 1 g of the reaction solution after passing through the catalyst charge tank was sampled and made into a homogeneous solution by adding 10 g of heated pure water. The concentration (in weight %) of the generated trioxane was measured by analyzing the solution by chromatograph. This concentration was used as the concentration of the target product in the presence of the styrene-based resin after operation. The reactivity change ratio (%) was then calculated from the following formula. If the reactivity change ratio was 70% or more, it was determined that the production can be performed while the decrease in intended reaction activity is suppressed even over prolonged use.

$$Reactivity change ratio ( \% ) = concentration ( in weight \% ) of target product in the presence of styrene - based resin after long - term operation / concentration ( in weight \% ) of target product in the presence of a styrene - based resin before long - term operation \times 100$$

### (3-4) Measurement of Retention Ratio of Ion Exchange Capacity Before and After Long-Term (Continuous) Operation

After the above-described production facility was operated for 270 days, the styrene-based resin was removed from the catalyst charge tank and rinsed with pure water. The ion exchange capacity of the styrene-based resin was measured by the following method, and the retention ratio of the ion exchange capacity before and after long-term operation was calculated.

### (Measurement of Ion Exchange Capacity)

Of the styrene-based resin, 20 mL was measured out and charged into a chromatography tube. Pure water was added up to the level of the top of the styrene-based resin. A receiving vessel was placed below the chromatography tube, and 500 mL of a 1-mol/L NaCl solution was poured into the chromatography tube. The cock of the chromatography tube was opened gradually so that the NaCl solution was made to flow at a rate of 500 mL/h. When the liquid level of the chromatography tube reached the level of the top of the styrene-based resin, the cock was closed. Using a 25-mL whole pipette, 25 mL of the liquid in the receiving vessel was measured out and 2 drops of a phenolphthalein solution was added as an indicator. The solution was titrated with a 0.1-mol/L NaOH solution until the color changed to light pink, and titration volume was determined. The ion exchange capacity (eq/L) of the styrene-based resin was calculated from the following formula

$$Ion exchange capacity ( eq / L ) = 0 \text{.1} \times ( factor of 0.1 - mol / L NaOH solution ) \times titration volume ( mL )$$

The retention ratio of the ion exchange capacity (%) of the styrene-based resin was calculated from the following equation.

$$Retention ratio of ion exchange capacity ( \% ) = ion exchange capacity after long - term operation ( eq / L ) / ion exchange capacity before long - term operation ( eq / L ) \times 100$$

### (3-5) Mechanical Strength Change Ratio Before and After Long-Term Operation (%)

After the above-described production facility was operated for 270 days, the styrene-based resin was removed from the catalyst charge tank and rinsed with pure water. The mechanical strengths of the styrene-based resin before and after the long-term operation were measured using a load displacement measurement apparatus under the following conditions.

### (Measurement Conditions for Load Displacement)

Load displacement measurement apparatus: Force gauge (ZTA-5N)
Test rate: 0.5 mm/min
A particulate sample was dried under vacuum and was placed on the measurement stage. The sample was compressed with a flat surface from above. The maximum load value (N) under which the sample was crushed was determined. The test was conducted on about 50 particles and the average value was calculated.

The mechanical strength change ratio (%) was calculated from the following formula. If the mechanical strength change ratio was 70% or more, it was determined that the styrene-based resin can be produced while the decrease in mechanical strength is reduced even over prolonged use.

$$Mechanical strength change ratio ( \% ) = ( maximum load value after long - term operation ) / ( maximum load value before long - term operation ) \times 100$$

### Production Example 1: Production of Styrene-Based Resin 1

Mixture 1 was prepared by mixing 360 g of styrene, 65 g of divinylbenzene, and 3.8 g of benzoyl peroxide. Subsequently, an aqueous solution was prepared by dissolving 27 g of sodium chloride and 3 g of ammonium salt of styrene-maleic anhydride copolymer in 722 g of water. Mixture 1 was then added to the aqueous solution to obtain Mixture 2. After Mixture 2 was stirred at 100 rpm, the organic phase was dispersed to form fine droplets. Mixture 2 was heated to 88 [symbol] C for 6 hours to take place a polymerization reaction. Then, 0.5 g of benzoyl peroxide was added to Mixture 2, and Mixture 2 was heated to 94 [symbol] C for another 1 hour. Thereafter, the resulting beads were filtered, washed, and dried at 125 [symbol] C for 5 hours. A white opaque polymer weighing 421 g was obtained in the form of spherical or ellipsoidal particles. While stirring, 70 g of the resulting polymer and 800 g of sulfuric acid were heated to 120 [symbol] C for 7 hours. The mixture containing the polymer was then cooled to about 35 [symbol] C and diluted with water. The solid product was filtered, washed with water to remove water-soluble products, and filtered. A styrene-based resin (cation exchange resin) weighing 273 g as the sulfonated product was yielded.

### Production Example 2: Production of Styrene-Based Resin 2

Mixture 1 was prepared by mixing 360 g of styrene, 65 g of divinylbenzene, 3.8 g of benzoyl peroxide, and 400 g of 2-butanol. Subsequently, an aqueous solution was prepared by dissolving sodium chloride (27 g) and 3 g of ammonium salt of styrene-maleic anhydride copolymer in 722 g of water. Mixture 1 was then added to the aqueous solution to prepare Mixture 2. After Mixture 2 was stirred at 100 rpm, the organic phase was dispersed to form fine droplets. Mixture 2 was heated to 88° C. for 6 hours to take place a polymerization reaction. Then, 0.5 g of benzoyl peroxide was added to Mixture 2, and Mixture 2 was heated to 94 IC for another 1 hour. Steam was then passed through the reaction mixture to remove the azeotropic mixture of 2-butanol and water. Thereafter, the resulting beads were filtered, washed, and dried at 125 DC for 5 hours. A white opaque polymer weighing 421 g was obtained in the form of spherical or ellipsoidal particles. Using the same method as in Production Example 1, a styrene-based resin (cation exchange resin) weighing 270 g as the sulfonated product was yielded.

### Production Example 3: Production of Styrene-Based Resin 3

The process was carried out in the same manner as in Production Example 2 except that Production Example 2 was changed as follows: 72 g of divinylbenzene and 500 g of 2-butanol were used and the polymer weighing 428 g was obtained, to yield a styrene-based resin (cation exchange resin) weighing 272 g as the sulfonated product.

### Production Example 4: Production of Styrene-Based Resin 4

The process was carried out in the same manner as in Production Example 1 except that Production Example 1 was changed as follows: 162 g of divinylbenzene was used and the polymer weighing 517 g was obtained, to yield a styrene-based resin (cation exchange resin) weighing 271 g as the sulfonated product.

### Production Example 5: Production of Styrene-Based Resin 5

The process was carried out in the same manner as in Production Example 1 except that Production Example 1 was changed as follows: 29 g of divinylbenzene was used and the polymer weighing 385 g was obtained, to yield a styrene-based resin (cation exchange resin) weighing 274 g as the sulfonated product.

### Production Example 6: Production of Styrene-Based Resin 6

The process was carried out in the same manner as in Production Example 1 except that Production Example 1 was changed as follows: 120 g of divinylbenzene was used and the polymer weighing 475 g was obtained, to yield a styrene-based resin (cation exchange resin) weighing 274 g as the sulfonated product.

### Production Example 7: Production of Styrene-Based Resin 7

The process was carried out in the same manner as in Production Example 1 except that Production Example 1 was changed as follows: 29 g of divinylbenzene was used, the polymerization temperature after adding benzoyl peroxide was maintained at 88 [symbol] C, and the polymer weighing 385 g was obtained, to yield a styrene-based resin (cation exchange resin) weighing 274 g as the sulfonated product.

### Production Example 8: Production of Styrene-Based Resin 8

The process was carried out in the same manner as in Production Example 2 except that 500 g of sulfuric acid was used in the sulfonation in Production Example 2, to yield a styrene-based resin (cation exchange resin) weighing 270 g as the sulfonated product.

### Production Example 9: Production of Styrene-Based Resin 9

The process was carried out in the same manner as in Production Example 2 except that Production Example 2 was changed as follows: 500 g of 2-butanol was used and the polymer weighing 421 g was yielded. The process was carried out in the same manner as in Production Example 2 except that 500 g of sulfuric acid was used in sulfonation, to yield a styrene-based resin (cation exchange resin) weighing 268 g as the sulfonated product.

### Production Example 10: Production of Styrene-Based Resin 10

The process was carried out in the same manner as in Production Example 1 except that Production Example 1 was changed as follows: 180 g of divinylbenzene was used and the polymer weighing 535 g was obtained, to yield a styrene-based resin (cation exchange resin) weighing 271 g as the sulfonated product.

### Production Example 11: Production of Styrene-Based Resin 11

The process was carried out in the same manner as in Production Example 1 except that Production Example 1 was changed as follows: 18 g of divinylbenzene was used and the polymer weighing 374 g was obtained, to yield a styrene-based resin (cation exchange resin) weighing 273 g as the sulfonated product.

### Production Example 12: Production of Styrene-Based Resin 12

The process was carried out in the same manner as in Production Example 1 except that Production Example 1 was changed as follows: the polymerization temperature was 78 [symbol] C and the polymer weighing 383 g was obtained, to yield a styrene-based resin (cation exchange resin) weighing 135 g as the sulfonated product.

### Production Example 13: Production of Styrene-Based Resin 13

The process was carried out in the same manner as in Production Example 1 except that Production Example 1 was changed as follows: benzoyl peroxide1 was not added to Mixture 2 and the polymer weighing 383 g was obtained, to yield a styrene-based resin (cation exchange resin) weighing 274 g as the sulfonated product.

### Production Example 14: Production of Styrene-Based Resin 14

The process was carried out in the same manner as in Production Example 1 except that Production Example 1 was changed as follows: 120 g of divinylbenzene was used, the stirring speed was 20 rpm, and the polymer weighing 432 g was obtained, to yield a styrene-based resin (cation exchange resin) weighing 274 g as the sulfonated product.

### Production Example 15: Production of Styrene-Based Resin 15

The process was carried out in the same manner as in Production Example 1 except that Production Example 1 was changed as follows: 120 g of divinylbenzene was used, the polymerization temperature was 78 [symbol] C, benzoyl peroxide was not added to Mixture 2, the stirring speed was 20 rpm, and the polymer weighing 384 g was obtained, to yield a styrene-based resin (cation exchange resin) weighing 274 g as the sulfonated product.

In the styrene-based resins of Examples 1 to 15, R¹, R², and R³ were hydrogen atoms, X was —SO₃H, Y was a single bond, a was an integer from 1 to 5, and b was an integer from 1 to 4 in the formula (I) and formula (II). The styrene-based resin of Production Example 10 had a higher degree of cross-linking than the styrene-based resin of Production Example 1. The styrene-based resin of Production Example 11 had a lower degree of cross-linking than the styrene-based resin of Production Example 1.

### Examples 1-10

In Examples 1 to 5, 6, 8, and 10, trioxane was synthesized using Styrene-based resins 1 to 5, 8, 6, and 7, respectively, and evaluated as described in “(2) Ratios of changes in characteristics before and after long-term test”. In Example 7, each evaluation was carried out in the same manner as in Example 1 except that the charge volume of the styrene-based resin in the long-term test was changed to 230 mL. In Example 9, each evaluation was carried out in the same manner as in Example 8 except that the operation of removing the supernatant liquid four times was not performed in the (2-1) long-term test. The evaluation results are summarized in Table 1.

### Comparative Examples 1-7

In Comparative Examples 1 to 3, 4, and 5 to 7, each evaluation was carried out in the same manner as in Example 1 except that Styrene-based resins 10 to 12, 7, and 13 to 15 were used instead of Styrene-based resin 1, respectively, and the operation of removing the supernatant liquid four times was not performed in the (2-1) long-term test. The evaluation results are summarized in Table 2.

### Example 11

Each evaluation was carried out in the same manner as in Example 1 except that methylal was synthesized using a mixture of formaldehyde and methanol as the raw material. The evaluation results are summarized in Table 1.

### Comparative Examples 8-9

In Comparative Examples 8 and 9, each evaluation was carried out in the same manner as in Example 11 except that Styrene-based resins 10 and 11 were used instead of Styrene-based resin 1, respectively. The evaluation results are summarized in Table 2.

TABLE 1

| | | Exam- | Exam- | Exam- | Exam- | Exam- | Exam- | Exam- | Exam- | Exam- | Exam- | Exam- |
|---|---|---|---|---|---|---|---|---|---|---|---|---|
| | | ple 1 | ple 2 | ple 3 | ple 4 | ple 5 | ple 6 | ple 7 | ple 8 | ple 9 | ple 10 | ple 11 |
| Target product | | T | T | T | T | T | T | T | T | T | T | M |
| Styrene- | Number | 1 | 2 | 3 | 4 | 5 | 8 | 1 | 6 | 6 | 7 | 1 |
| based | Addition of polymerization | Yes | Yes | Yes | Yes | Yes | Yes | Yes | Yes | Yes | Yes | Yes |
| resin | initiator | | | | | | | | | | | |
| | Temperature rise after addition | Yes | Yes | Yes | Yes | Yes | Yes | Yes | Yes | Yes | No | Yes |
| | of polymerization initiator | | | | | | | | | | | |
| | Stirring speed (rpm) | 100 | 100 | 100 | 100 | 100 | 100 | 100 | 100 | 100 | 100 | 100 |
| Treatment with formaldehyde solution prior | | Yes | Yes | Yes | Yes | Yes | Yes | Yes | Yes | No | Yes | Yes |
| to reaction | | | | | | | | | | | | |
| Peak area ratio (A1/A2) | | 1.12 | 1.12 | 1.14 | 1.39 | 1.02 | 1.12 | 1.12 | 1.29 | 1.36 | 1.35 | 1.12 |
| Charge ratio (%) of catalyst charge tank | | 50 | 50 | 50 | 50 | 50 | 50 | 92 | 50 | 50 | 50 | 50 |
| Residence time of formaldehyde (minutes) | | 6.9 | 6.9 | 6.9 | 6.9 | 6.9 | 6.9 | 6.9 | 6.9 | 6.9 | 6.9 | 5.8 |
| Evaluation | Volume change ratio (%) before | 115 | 128 | 142 | 110 | 140 | 128 | 105 | 120 | 112 | 115 | 110 |
| results | and after long-term test | | | | | | | | | | | |
| | Reactivity change ratio (%) | 83 | 73 | 89 | 72 | 90 | 75 | 71 | 75 | 73 | 74 | 85 |
| | before and after long-term test | | | | | | | | | | | |

In the target products in the table, “T” represents trioxane, and “M” represents methylal.

TABLE 2

| | | Comp. | Comp. | Comp. | Comp. | Comp. | Comp. | Comp. | Comp. | Comp. |
|---|---|---|---|---|---|---|---|---|---|---|
| | | Ex. 1 | Ex. 2 | Ex. 3 | Ex. 4 | Ex. 5 | Ex. 6 | Ex. 7 | Ex. 8 | Ex. 9 |
| Target product | | T | T | T | T | T | T | T | M | M |
| Styrene- | Number | 10 | 11 | 12 | 7 | 13 | 14 | 15 | 10 | 11 |
| based | Addition of polymerization initiator | Yes | Yes | Yes | Yes | No | Yes | No | Yes | Yes |
| resin | Temperature rise after addition | Yes | Yes | Yes | No | Yes | Yes | No | Yes | Yes |
| | of polymerization initiator | | | | | | | | | |
| | Stirring speed (rpm) | 100 | 100 | 100 | 100 | 100 | 20 | 20 | 100 | 100 |
| Treatment with formaldehyde solution prior to reaction | | Yes | Yes | Yes | Yes | Yes | Yes | Yes | Yes | Yes |
| Peak area ratio (A1/A2) | | 1.42 | 0.98 | 1.41 | 1.42 | 1.43 | 1.42 | 1.47 | 1.42 | 0.98 |
| Charge ratio (%) of catalyst charge tank | | 50 | 50 | 50 | 50 | 50 | 50 | 50 | 50 | 50 |
| Residence time of formaldehyde (minutes) | | 6.9 | 6.9 | 6.9 | 6.9 | 6.9 | 6.9 | 6.9 | 5.8 | 5.8 |
| Evaluation | Volume change ratio (%) before and | 104 | 154 | 103 | 105 | 103 | 104 | 102 | 102 | 152 |
| results | after long-term test | | | | | | | | | |
| | Reactivity change ratio (%) before | 68 | 90 | 68 | 67 | 65 | 66 | 63 | 68 | 90 |
| | and after long-term test | | | | | | | | | |

In the target products in the table, “T” represents trioxane, and “M” represents methylal.

### Examples 12 to 15

In Examples 12, 13, 14, and 15, trioxane was synthesized using Styrene-based resins 1, 2, 8, and 9, respectively, and evaluated as described in “(3) Ratios of changes in characteristics before and after long-term operation”. The evaluation results are summarized in Table 3.

TABLE 3

| | | Example 12 | Example 13 | Example 14 | Example 15 |
|---|---|---|---|---|---|
| Target product | | T | T | T | T |
| Styrene- | Number | 1 | 2 | 8 | 9 |
| based resin | Addition of polymerization initiator | Yes | Yes | Yes | Yes |
| | Temperature rise after addition of polymerization initiator | Yes | Yes | Yes | Yes |
| | Stirring speed (rpm) | 100 | 100 | 100 | 100 |
| Treatment with formaldehyde solution prior to reaction | | Yes | Yes | Yes | Yes |
| Peak area ratio (A1/A2) | | 1.12 | 1.12 | 1.12 | 1.12 |
| Charge ratio (%) of catalyst charge tank | | 50 | 50 | 50 | 50 |
| Residence time of formaldehyde (minutes) | | 6.9 | 6.9 | 6.9 | 6.9 |
| Evaluation | Retention ratio (%) of ion exchange resin before and after long-term operation | 10.5 | 15.2 | 21.7 | 7.4 |
| results | Volume change ratio (%) before and after long-term operation | 114 | 130 | 128 | 133 |
| | Reactivity change ratio (%) before and after long-term operation | 72 | 73 | 75 | 70 |
| | Mechanical strength change ratio (%) before and after long-term operation | 82 | 75 | 80 | 70 |

In the target products in the table, “T” represents trioxane, and “M” represents methylal.

The reduction in the reactivity change ratio of the styrene-based resin before and after the test was suppressed in Examples 1 to 15. In addition, the volume change ratio of the styrene-based resin before and after the test in Examples 1 to 15 was satisfactory.

On the other hand, the decrease in reaction activity could not be suppressed in Comparative Examples 1 and 3 to 8 because the area ratio (A1/A2) exceeded the upper limit of the present disclosure. In addition, the volume change of the styrene-based resin could not be suppressed in Comparative Examples 2 and 9 because the area ratio (A1/A2) was below the lower limit of the present disclosure.

### INDUSTRIAL APPLICABILITY

The present disclosure can provide a method for producing trioxane or methylal wherein the decrease in intended reaction activity is suppressed and the volume change of the styrene-based resin is suppressed even over prolonged use.

1. A method for producing trioxane or methylal comprising the step of
reacting formaldehyde in the presence of a styrene-based resin having an electron-withdrawing group to produce trioxane or methylal,
wherein a solid-state ¹³C-NMR spectrum of the styrene-based resin has Peak 1 having a peak top in a range of 32 to 57 ppm and Peak 2 having a peak top in a range of 120 to 137 ppm, and
an area ratio (A1/A2) of an area A1 of Peak 1 to an area A2 of Peak 2 is 1.00 to 1.40.

2. The method for producing trioxane or methylal according to claim 1, wherein the styrene-based resin is a cation exchange resin.

3. The method for producing trioxane or methylal according to claim 1, wherein the styrene-based resin comprises a constituent unit represented by the following formula (I) and a constituent unit represented by the following formula (II): 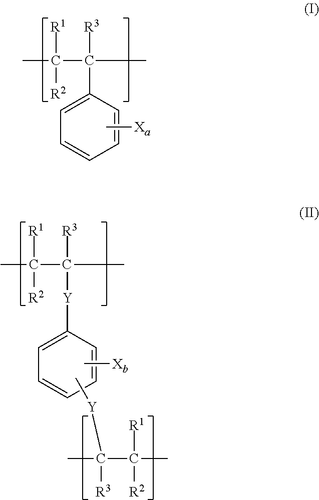
in the formulae (I) and (II),
R¹, R², and R³ are each independently a hydrogen atom, an alkyl group, an aryl group, or a halogen atom;
X is an electron-withdrawing group, and the plurality of X may be different from each other;
Y is an alkylene group, an arylene group, or a single bond;
a is each independently an integer from 1 to 5;
b is each independently an integer from 1 to 4.

4. The method for producing trioxane or methylal according to claim 1, wherein
a catalyst charge tank charged with the styrene-based resin is used, and
a charge ratio of the styrene-based resin in the catalyst charge tank is 20 to 90% of a volume of a chargeable portion in the catalyst charge tank.

5. The method for producing trioxane or methylal according to claim 1, wherein the reaction of formaldehyde in the step is a continuous reaction.

6. The method for producing trioxane or methylal according to claim 4, wherein a residence time of formaldehyde in the catalyst charge tank is 0.01 to 50 minutes.

7. The method for producing trioxane or methylal according to claim 4, further comprising a step (P) of passing an aqueous solution of formaldehyde in a volume equal to or greater than a charge volume of the styrene-based resin, through the catalyst charge tank before the step.
